(19) United States
(12) Patent Application Publication
(10) Pub. No.: US 2020/0093764 A1
(43) Pub. Date: Mar. 26, 2020

(54) TREATMENT OF ASTHMA WITH CYSTEAMINE

(71) Applicant: CHILDREN'S HOSPITAL MEDICAL CENTER, Cincinnati, OH (US)

(72) Inventors: Gurjit K. HERSHEY, Cincinnati, OH (US); Jocelyn BIAGINI-MYERS, Cincinnati, OH (US); Hong JI, Blue Ash, OH (US); Lisa J. MARTIN, West Chester, OH (US)

(73) Assignee: CHILDREN'S HOSPITAL MEDICAL CENTER, Cincinnati, OH (US)

(21) Appl. No.: 16/484,052

(22) PCT Filed: Feb. 7, 2018
(86) PCT No.: PCT/US2018/017183

§ 371 Date: Aug. 6, 2019

Related U.S. Application Data
(60) Provisional application No. 62/455,661, filed on Feb. 7, 2017.
(60) Provisional application No. 62/579,406, filed on Oct. 31, 2017.

(51) Int. Cl. A61K 31/145; A61K 31/573; A61P 11/06

(52) U.S. Cl. CPC A61K 31/145; A61K 9/0019; A61P 11/06; A61K 31/573

(57) ABSTRACT

Provided herein are cysteamine or a pharmaceutically acceptable salt thereof and methods of using such for treating asthma (e.g., moderate to severe persistent asthma) or reducing the risk of asthma occurrence.

### CROSS-REFERENCE TO RELATED APPLICATIONS

This application claims the benefit under 35 U.S.C. § 119(e) of U.S. provisional application Nos. 62/455,661 filed Feb. 7, 2017 and 62/579,406 filed Oct. 31, 2017, the contents of which are incorporated by reference herein in their entirety.

### GOVERNMENT SUPPORT

This invention was made with government support under Contract No. NIH AI 070235 awarded by National Institutes of Health. The government has certain rights in the invention.

### BACKGROUND

Asthma affects 25.7 million people in the US including 7.0 million children. Akinbami et al., NCHS data brief 2012:1-8. Although patients suffering from asthma share similar clinical symptoms, the disease is heterogeneous. Bel, The New England journal of medicine 2013; 369:2362. This heterogeneity contributes to the difficulty in both studying and treating asthma. Nearly two-thirds of children who currently have asthma reported at least one attack in the previous 12 months (Fassl et al., Pediatrics 2012; 130:482-91), highlighting the suboptimal management of asthma in this age group (Akinbami, Advance data 2006:1-24). Up to 27% of children admitted for asthma exacerbation require longer than a three-day stay and this phenotype seems to be conserved within a given individual and is partially heritable. Akinbami, 2006; and Morray et al., Archives of pediatrics & adolescent medicine 1995; 149:276-9. Thus, this may represent a distinct phenotype of asthma that is poorly responsive to standard treatment regimens for inpatient asthma. Further, the frequency of absent or incomplete efficacy in asthma treatment (e.g., a steroid treatment) has been estimated to be 40-70%. Drazen et al., British medical bulletin 2000; 56:1054-70. Accordingly, there is a need for alternative non-steroid-based asthma treatment.

### SUMMARY OF THE INVENTION

The present disclosure is, at least in part, based on the unexpected discoveries of the prophylactic effect of cysteamine against asthma development in subjects at risk for asthma, and also therapeutic effect of cysteamine on reducing asthma symptoms in asthmatic subjects, e.g., subjects with moderate to severe persistent asthma. Further, it was discovered that such prophylactic and/or therapeutic effects on asthma treatment were achieved at cysteamine doses that are significantly lower than that currently used for treatment of cystinosis.

Accordingly, one aspect of the present disclosure features a method for treating asthma or reducing the risk of asthma occurrence by administering an effective amount of cysteamine or a pharmaceutically acceptable salt thereof. For example, the subject can be a human patient at risk for asthma or a human patient having moderate to severe persistent asthma.

In another aspect, the present disclosure provides a method for treating asthma by administering to the subject in need thereof cysteamine or a pharmaceutically acceptable salt thereof in an amount equivalent to 5 mg/kg/day to 25 mg/kg/day of free base cysteamine.

In any of the methods described herein, the subject in need of cysteamine or a pharmaceutically acceptable salt thereof can be a human patient at risk for asthma, e.g., for a prophylactic treatment, or a human patient having moderate to severe persistent asthma, e.g., for a therapeutic treatment. The subject to be treated can be an adult or a children. In some embodiments, the subject to be treated is an adult. In some embodiments, the subject to be treated is a child at the age of 12 or over. In some embodiments, the subject to be treated is a child at the age of 5-11. In some embodiments, the subject to be treated is a child under the age of 5.

The cysteamine or a pharmaceutically acceptable salt thereof can be administered to the subjects in need thereof in an effective amount to achieve a desirable clinical effect. For example, a human patient having moderate to severe persistent asthma can be administered cysteamine or a pharmaceutically acceptable salt thereof in an amount sufficient to reduce asthma exacerbation, e.g., induced by allergen. As another example, a human patient at risk for asthma can be administered cysteamine or a pharmaceutically acceptable salt thereof in an amount sufficient to reduce the risk of asthma occurrence or development. In some embodiments, the effective amount of the cysteamine or the pharmaceutically acceptable salt thereof administered to a subject in need thereof may be equivalent to 5 mg/kg/day to 25 mg/kg/day of free base cysteamine, 6.25 mg/kg/day to 25 mg/kg/day of free base cysteamine, or 5 mg/kg/day to 15 mg/kg/day of free base cysteamine. In some embodiments, the effective amount of the cysteamine or the pharmaceutically acceptable salt thereof administered to a subject in need thereof may be equivalent to: (i) 200 mg/day to 500 mg/day of free base cysteamine for a subject over the age of 12 and/or over 50 kg in body weight; (ii) 120 mg/day to 450 mg/day of free base cysteamine for a subject having a body weight in a range of 20 kg to 50 kg; and (iii) 40 mg/day to 250 mg/day of free base cysteamine for a subject having a body weight under 20 kg. The daily effective amount as described herein can be administered to the subjects once a day or divided into multiple doses to be administered, e.g., 2-4 times a day.

The cysteamine or the pharmaceutically acceptable salt thereof can be administered to the subject via any administration route, including, e.g., by oral administration or by injection. In some embodiments, the cysteamine or the pharmaceutically acceptable salt thereof can be formulated in an enteric-coated coated form and/or in a sustained-release form, e.g., suitable for oral administration.

Cysteamine for administration to subjects in need thereof can be provided in a free base form, in a disulfide form, or in a pharmaceutically acceptable salt form. Examples of cysteamine include, but are not limited to cysteamine bitartrate, cysteamine hydrochloride, and cystamine.

Also within the scope of the present disclosure are (i) a pharmaceutical composition for use in treating asthma in a subject, the composition comprising cysteamine in any suitable form as described herein and a pharmaceutically acceptable carrier; and (ii) use of cysteamine in any suitable form as described herein in manufacturing a medicament for use in treating asthma in a subject. The subject can be at risk for asthma, or having asthma (e.g., with moderate to severe persistent asthma).

The details of one or more embodiments of the disclosure are set forth in the description below. Other features or advantages of the present disclosure will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1C are diagrams showing that cysteamine prophylactic treatment significantly prevented asthma in VNN1 KO and normal mice. Cysteamine or cystamine treatment blocks airway hyper-responsiveness (AHR) development in mice when administered through oral or IP methods. FIG. 1A: a schematic illustration showing the treatment dosage for both VNN1 KO and WT Balb/c mice over a 24 hour period. FIG. 1B: a diagram showing the effect of treatments on VNN1 KO mice. FIG. 1C: a chart showing the effect of treatment on WT Balb/c mice.

FIGS. 2A-2H are diagrams showing that cysteamine treatment significantly decreased asthma, including AHR and airway inflammation, in mice. FIG. 2A: a schematic illustration showing the treatment dosage for WT Balb/c mice over a 24 hour period. FIG. 2B: a chart showing the resistance of WT-HDM, WT-HDM+Cyst, and SAL WT mice treated with various doses of Methacholine. FIG. 2C: a diagram showing the resistance of WT-HDM, WT-HDM+Cyst, and SAL WT mice treated with 25 mg/ml of Methacholine. FIG. 2D: a diagram showing BALF cell (×10⁴) concentration of WT-HDM, WT-HDM+Cyst, and SAL WT mice. FIG. 2E: a diagram showing the Eosinophils (×10³) concentration of WT-HDM, WT-HDM+Cyst, and SAL WT mice. FIG. 2F: a diagram showing CD44⁺ IL-13⁺ (×10³) concentration of WT-HDM, WT-HDM+Cyst, and SAL WT mice. FIG. 2G: a diagram showing CD44⁺ IL-17A⁺ (×10³) concentration of WT-HDM, WT-HDM+Cyst, and SAL WT mice. FIG. 2H: a diagram showing CD44⁺ IFNγ⁺ (×10³) concentration of WT-HDM, WT-HDM+Cyst, and SAL WT mice.

FIGS. 3A-3D are diagrams showing the effectiveness of Cystagon® (cysteamine bitartrate) at blocking AHR in mice with asthma. The 6.25 mg/kg/day dose was effective at reducing AHR, but maximum effectiveness was achieved at 12.5 mg/kg/day dose which is one fourth of the dose used for treating cystinosis. FIG. 3A: a schematic illustration showing the treatment dosage for WT BALB/c mice. FIG. 3B: a chart showing the resistance of HDM, HDM+6.25, HDM+12.5, HDM+25, HDM+50 mice over various dosages of Methacholine. FIG. 3C: a chart showing the resistance concentration of HDM, HDM+6.25, HDM+12.5, HDM+25, HDM+50 mice after 50 mg/mL of Methacholine. FIG. 3D: a diagram showing that Cystagon® (cysteamine bitartrate) treatment reduced airway inflammation

FIG. 4A is a table showing drug kinetics in mouse serum after administration of cysteamine.

FIG. 4B is a chart showing the cysteamine concentrations in μmol/L over the time after the ends of cysteamine infusion in minutes.

FIG. 5A is a diagram showing that cysteamine treatment decreased AHR similarly to dexamethasone treatment in wild-type (WT) mice.

FIG. 5B is a diagram showing cysteamine treatment significantly decreased the presence of Th1, Th2, and Th17 cells present in the lungs of wild-type (WT) mice. There were no significant differences between treatment groups.

FIGS. 6A-6E are diagrams showing that oral cysteamine treatment was equivalent to IP injection. FIG. 6A is a schematic illustration showing the treatment dosage for wild-type (WT) Balb/c mice. FIG. 6B is a chart showing the resistance of HDM/HDM, HDM/HDM+6.25, Saline/HDM, and HDM/HDM+12.5 mice over various dosages of Methacholine. FIG. 6C is a chart showing the concentration of resistance after 50 mg/mL of methacholine in the various treatment groups. FIG. 6D is a diagram showing the concentration of BALF Counts (×10³) in the various treatment groups. FIG. 6E is a diagram showing the concentration of lung cells (×10³) in the various treatment groups.

FIG. 7 is a schematic illustration showing an exemplary design of an adaptive study with dose escalation following analysis of side effects and evidence of efficacy over the course of 5 months.

### DETAILED DESCRIPTION OF THE INVENTION

Asthma affects a large number of people in the U.S., including children. Although patients suffering from asthma share similar clinical symptoms, the disease is heterogeneous, which contributes to the difficulty in both studying and treating asthma. About 40-70% of asthmatic patients do not respond to currently available therapies, e.g., a steroid therapy. Accordingly, there is a need to develop a non-steroid-based methods and compositions for treatment of asthma.

The present disclosure is, at least in part, based on the unexpected discovery of the prophylactic effect of cysteamine against asthma development in subjects who are at risk for asthma. It was also unexpectedly discovered that cysteamine alone (without a steroid treatment, which is currently the standard treatment for asthma) can effectively reduce asthma symptoms, e.g., reduced airway inflammation and/or responsiveness to an allergen, in asthmatic subjects, e.g., subjects with moderate to severe persistent asthma. Further, it was discovered that such prophylactic and/or therapeutic effects on asthma treatment can be achieved with cysteamine doses that are significantly lower than that used for treatment of cystinosis. For example, it was shown that a dose of cysteamine that is one-fourth of the dose of cysteamine for treatment of cystinosis or lower was effective to reduce asthma symptoms, e.g., reduced airway inflammation and/or responsiveness to an allergen.

Accordingly, described herein are methods and compositions for treating asthma or reducing the risk of asthma occurrence in subjects using cysteamine or a pharmaceutically acceptable salt thereof. Such treatment, which may involve low doses of cysteamine, may address the side effects concerns associated with cysteamine treatment (e.g., nausea or dyspepsia). When applied to human patients at risk for asthma, the treatment methods described herein would be expected to achieve prophylactic effects. Further, the treatment methods described herein would be particularly effective in treating human patients having moderate to severe persistent asthma and/or reduce asthma exacerbation induced by allergens (e.g., novel allergens to which the subject has not been exposed previously).

In some aspects, the disclosure relates to methods for treating asthma or reducing the risk of asthma occurrence using cysteamine or a pharmaceutically acceptable salt thereof, which can be provided as a prophylactic or therapeutic treatment for asthma in subjects in need thereof.

### I. Cysteamine and Pharmaceutical Compositions Comprising the Same

Cysteamine is an amino thiol with the chemical formula HSCH₂CH₂NH₂. Cysteamine is also known as β-mercaptoethylamine, 2-aminoethanethiol, 2-mercaptoethylamine, decarboxycysteine, thioethanolamine and mercaptamine. Cysteamine bitaritrate (Cystagon®) is approved by the U.S. Food and Drug Administration (FDA) for treatment of cystinosis, which is an autosomal recessive metabolic disorder caused by mutations in the cystinosis, nephropathic (CTNS) gene encoding the lysosomal cystine carrier cystinosin. The disease is characterized by lysosomal cystine accumulation and initially damages the kidneys, later affecting the eyes, endocrine organs, and neuromuscular system. Cysteamine hydrochloride (Cystaran®) is approved by the FDA for the treatment of corneal cystine crystal accumulation in patients with cystinosis. Further, cysteamine has been used for preserving renal function, enhancing growth, and postponing extrarenal complications in patients with cystinosis. Cysteamine enters lysosomes and converts cystine into cysteine and cysteine-cysteamine mixed disulfide, both of which can exit the lysosome.

As described herein, cysteamine for use in the methods described herein may be in a free base form (i.e., HSCH₂CH₂NH₂), in a dimer or multimer form, for example, in a disulfide form (e.g., cystamine (NH₂CH₂CH₂S—SCH₂CH₂NH₂)) or in a salt form. Appropriate pharmaceutically acceptable salts of cysteamine include salts of organic acids, e.g., carboxylic acids, including but not limited to acetate, trifluoroacetate, lactate, gluconate, citrate, tartrate, maleate, malate, pantothenate, isethionate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-napthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. Cysteamine may in some cases be isolated as the hydrate. Examples of pharmaceutically acceptable salt of cysteamine include, but are not limited to cysteamine hydrochloride (HCl) (C₂H₇NS.HCl, 1 mg=0.7 mg free cysteamine base), phosphocysteamine sodium salt (e.g., C₂H₇NO₃PSNa, 1 mg=0.4 mg free cysteamine base) and cysteamine bitartrate (C₂H₇NS.C₄H₆O₆, 1 mg=0.3 mg free cysteamine base).

Cysteamine, in any of the suitable forms described herein, or a pharmaceutically acceptable salt thereof, may be formulated with one or more pharmaceutically acceptable carrier, diluent, and/or excipient to form a pharmaceutical composition. A carrier, diluent or excipient that is “pharmaceutically acceptable” includes one that is sterile and pyrogen free. Suitable pharmaceutical carriers, diluents and excipients are well known in the art. The carrier(s) must be “acceptable” in the sense of being compatible with the inhibitor and not deleterious to the recipients thereof.

A pharmaceutical composition comprising cysteamine (e.g., in any suitable form as described herein) or a pharmaceutically acceptable salt thereof can be formulated according to routes of administration, including, e.g., parenteral administration, oral administration, buccal administration, sublingual administration, topical administration, or inhalation.

In some embodiments, the pharmaceutical composition or formulation is suitable for oral, buccal or sublingual administration, such as in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavoring agents, coloring matter or dyes, with emulsifying and/or suspending agents and with diluents such as water, ethanol, propylene glycol and glycerin, and combinations thereof.

In some embodiments, the pharmaceutical compositions are formulated in an enteric-coated solid form (e.g., but not limited to tablets, pellets, granules, which may be filled into capsule shells). An enteric coating may be a polymer barrier applied on a solid form that prevents its dissolution or disintegration in the gastic environment, thereby protecting active ingredients from the acidity of the stomach and/or also reducing gastrointestinal side effects. In some embodiments, the enteric coating does not dissolve in the gastric acid of the stomach (pH˜3), but begins to dissolve at a higher pH (e.g., a pH within the small intestine). In some embodiments, the enteric coating begins to dissolve or rapidly dissolves in an aqueous solution at pH between about 4.5 to about 5.5. In some embodiments, the enteric coating begins to dissolve or rapidly dissolves in an aqueous solution at pH between about 5.5 to about 7. In some embodiments, the enteric coating begins to dissolve or rapidly dissolves in an aqueous solution at pH between about 7 to about 9.

In some embodiments, the pharmaceutical compositions are formulated in a sustained-release form or delayed-release form. The sustained-release form or delayed-release form can be formulated such that it enables a q.d. (once a day) or b.i.d (twice a day) administration. In some embodiments, the sustained-release or delayed-release form may be formulated as a bead dosage form, e.g., as described in the PCT Publication No. WO 2014/204881, the relevant disclosures of which are incorporated by reference for the purposes or subject matter referenced herein.

In some embodiments, the pharmaceutical compositions or formulations are for parenteral administration, such as intravenous, intra-arterial, intra-muscular, subcutaneous, or intraperitoneal administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

In some embodiments, the pharmaceutical composition or formulation is suitable for intranasal administration or inhalation, such as delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoro-ethane, a hydrofluoroalkane, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant. Capsules and cartridges (made, for example, from gelatin) for use in an inhaler or insufflator may be formulated to contain a powder mix of the inhibitor and a suitable powder base such as lactose or starch.

The formulations may be presented in unit-dose or multi-dose containers, for example sealed ampoules or vials, and may be stored in a freeze-dried (lyophilized) condition requiring only the addition of the sterile liquid carrier immediately prior to use.

In some embodiments, the formulations can be pre-loaded in a unit-dose injection device, e.g., a syringe, for intravenous injection.

### II. Subjects

In some embodiments, a subject to be treated by any of the methods described herein can be a mammal, e.g., a human, having, suspected of having, or at risk for asthma. In some embodiments, the subject is a human patient suffering from an asthma exacerbation, also known as asthma attack, such as an acute asthma attack.

Asthma is an inflammatory disease of the airways. Common symptoms include wheezing, coughing, chest tightness, and shortness of breath. The severity and recurrence of symptoms vary between subjects. Asthma may also be classified as atopic (extrinsic) or non-atopic (intrinsic) where atopy refers to a predisposition toward developing type 1 hypersensitivity reactions. A subject having asthma may be diagnosed based on clinically available tests and/or an assessment of the pattern of symptoms in a subject and response to therapy. An exemplary available diagnostic test for asthma is spirometry. Spirometry is a lung function test that measures the volume and/or flow of air that can be inhaled and exhaled by a subject. However, spirometry may not be possible in younger children (e.g., under the age of 5).

There are generally three main results from spirometry: (1) FEV₁, which represents how much air a subject can forcefully exhale in one second; (2) FVC, which represents the maximum amount of air a subject can forcefully exhale; and (3) FEV1/FVC, which represents the percentage a subject's total air capacity (“vital capacity”) that the subject can forcefully exhale in one second. Spirometry results can be given in “liters per minute” or as a percentage of a reference value, e.g., a value obtained from a population of healthy normal subjects, e.g., at a similar age, height, weight, gender, and/or race as the subject to be diagnosed. Spirometry may be part of a bronchial challenge test, which may involve assessing bronchial hyper-responsiveness to rigorous exercise, inhalation of cold/dry air, and/or a pharmaceutical agent such as methacholine or histamine. Diagnostic methods for asthma are known in the art (see, e.g., Expert Panel Report 3: Guidelines for the Diagnosis and Management of Asthma. NIH Publication Number 08-5846 ed, National Institutes of Health, 2007).

Asthma may be classified as intermittent, mild persistent, moderate persistent, and severe persistent. The severity can be determined, e.g., based on frequency of asthma occurrence, frequency of nighttime awakening, frequency of using rescue medication, degree of interference with normal activity, and lung function, e.g., measured using spirometry.

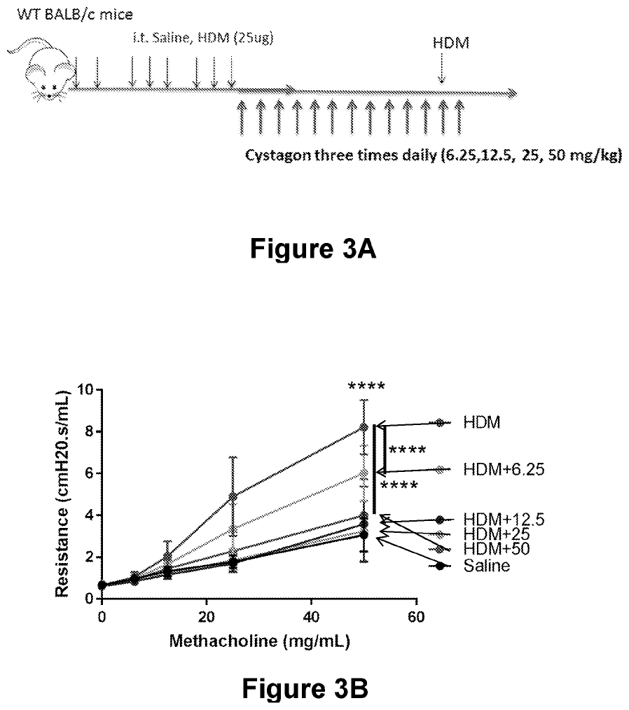

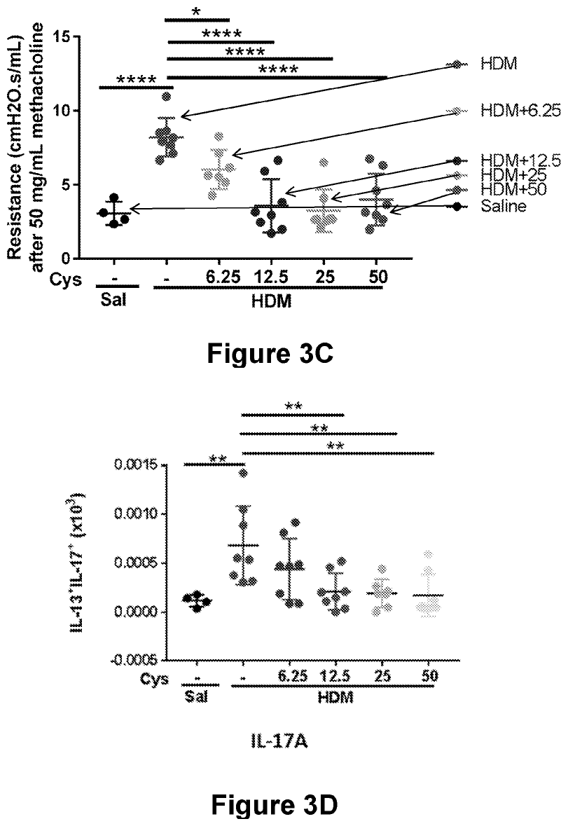

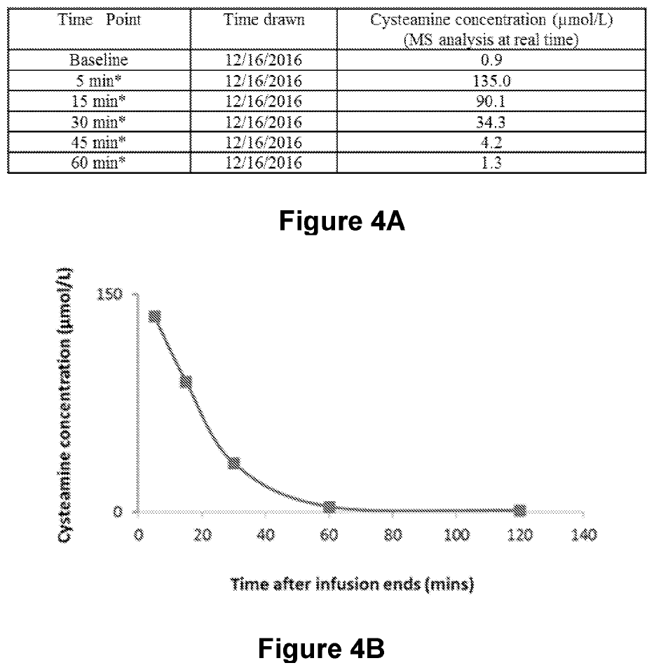

In some embodiments, subjects to be treated by the methods described herein can be subjects (e.g., human patients) having intermittent asthma. Diagnosis of intermittent asthma is generally known in the art, e.g., based on exemplary signs of intermittent asthma described in the National Heart, Lung, and Blood Institute. Expert panel report 3 (EPR-3): Guidelines for the diagnosis and management of asthma—Full Report 2007. FIGS. 3-4 a/b/c, pgs 72-74. For example, subjects with intermittent asthma usually have asthma symptoms two days per week or less. Their symptoms do not generally limit normal activity nor wake them up at night. However, subjects at the age of 5 or above, e.g., 5-80 years old, who have intermittent asthma, may be awakened at night two days/month or less because of asthma symptoms. They may use rescue medications, including, e.g., short-acting beta-agonists (SABA), two days per week or less. Subjects with intermittent asthma generally have normal or near-normal lung function, e.g., as assessed by spirometry. For example, older children and adults (e.g., subjects at the age of 5 or above, e.g., 5-80 years old), who have intermittent asthma, may exhibit a normal FEV₁ level between exacerbations. Additionally or alternatively, subjects at the age of 5-11, who have intermittent asthma, may exhibit a FEV₁ level that is at least 80% of a normal FEV₁ level and/or a FEV₁/FVC level that is greater than 85%. Subjects at the age of 12 or above (e.g., 12-80 years old), who have intermittent asthma, may exhibit a FEV₁ level that is greater than 80% of a normal FEV₁ level and/or a normal FEV₁/FVC value. A normal FEV₁ level or FEV₁/FVC value may be determined, e.g., based on a population of healthy normal subjects, e.g., at a similar age, height, weight, gender, and/or race as the subject to be diagnosed.

In some embodiments, subjects to be treated by the methods described herein can be subjects (e.g., human patients) having mild persistent asthma. Diagnosis of mild persistent asthma is generally known in the art, e.g., based on exemplary signs of mild persistent asthma described in the National Heart, Lung, and Blood Institute. Expert panel report 3 (EPR-3): Guidelines for the diagnosis and management of asthma—Full Report 2007. FIGS. 3-4 a/b/c, pgs 72-74. For example, subjects with mild persistent asthma generally have asthma symptoms more than twice a week, but not every day. Their daily activity is slightly limited due to asthma symptoms. Subjects with mild persistent asthma may wake up occasionally because of asthma symptoms. For example, younger children (e.g., under the age of 5) may wake up at night 1-2 times/month due to asthma symptoms, while older children and adults (e.g., subjects at the age of 5 or above) may wake up at night 3-4 times/month due to asthma symptoms. Subjects with mild persistent asthma generally use rescue medications more than twice a week to control their asthma symptoms, but not daily. However, for subjects at the age of 12 or above (e.g., 12-80 years), they may use rescue medication once a day if needed. Subjects with mild persistent asthma generally have near-normal lung function, e.g., as assessed by spirometry. For example, subjects at the age of 5-11, who have mild persistent asthma, may exhibit a FEV1 level that is greater than 80% of a normal FEV₁ level, and/or a FEV1/FVC value that is greater than 80%. Subjects at the age of 12 or above (e.g., 12-80 years old), who have mild persistent asthma, may exhibit a FEV₁ level that is equal to or greater than 80% of a normal FEV₁ level, and/or a normal FEV₁/FVC value. As described above, a normal FEV1 level or FEV1/FVC value may be determined, e.g., based on a population of healthy normal subjects, e.g., at a similar age, height, weight, gender, and/or race as the subject to be diagnosed.

In some embodiments, subjects to be treated by the methods described herein can be subjects (e.g., human patients) having moderate to severe persistent asthma. Diagnosis of moderate and severe persistent asthma is generally known in the art, e.g., based on exemplary signs of moderate and severe persistent asthma described in the National Heart, Lung, and Blood Institute. Expert panel report 3 (EPR-3): Guidelines for the diagnosis and management of asthma—Full Report 2007. FIGS. 3-4 a/b/c, pgs 72-74. For example, subjects with moderate persistent asthma generally have asthma symptoms every day. Their daily activity is somewhat limited by asthma symptoms. Older children and adults (e.g., at the age of 5 or above) with moderate persistent asthma may wake up at night at least once a week because of asthma symptoms, while younger children (e.g., under the age of 5) may wake up at night 3-4 times a month because of asthma symptoms. Subjects with moderate persistent asthma generally use rescue medications daily to control their asthma symptoms. Subjects with moderate persistent asthma generally have decreased lung function, e.g., as assessed by spirometry. For example, subjects at the age of 5-11, who have moderate persistent asthma, may exhibit a FEV₁ level that is 60-80% of a normal FEV₁ level, and/or a FEV₁/FVC value of 75%-80%. Subjects at the age of 12 or above (e.g., 12-80 years old), who have moderate persistent asthma, may exhibit a FEV₁ level that is greater than 60% but less than 80% of a normal FEV₁ level, and/or a FEV₁/FVC value reduced by 5%, as compared to a normal FEV₁/FVC value. As described above, a normal FEV₁ level or FEV₁/FVC value may be determined, e.g., based on a population of healthy normal subjects, e.g., at a similar age, height, weight, gender, and/or race as the subject to be diagnosed.

Similarly, severe persistent asthma can be diagnosed, e.g., based on exemplary signs of severe persistent asthma that are known in the art. For example, subjects with severe persistent asthma generally have asthma symptoms throughout each day. As a result, their daily activity is extremely limited. Older children and adults (e.g., subjects at the age of 5 or above) with severe persistent asthma may often wake up, e.g., as often as 7 times a week, because of asthma symptoms, while younger children (e.g., a children under the age of 5) may wake up at night more than once a week because of asthma symptoms. Subjects with severe persistent asthma must use rescue medications several times a day to control their asthma symptoms. Subjects with severe persistent asthma generally have severely decreased lung function, e.g., as assessed by spirometry. For example, subjects at the age of 5-11, who have severe persistent asthma, may exhibit a FEV₁ level that is less than 60% of a normal FEV1 level, and/or a FEV₁/FVC value of less than 75%. Subjects at the age of 12 or above (e.g., 12-80 years old), who have severe persistent asthma, may exhibit a FEV₁ level that is less than 60% of a normal FEV₁ level, and/or a FEV₁/FVC value reduced by greater than 5%, as compared to a normal FEV₁/FVC value. As described above, a normal FEV₁ level or FEV₁/FVC value may be determined, e.g., based on a population of healthy normal subjects, e.g., at a similar age, height, weight, gender, and/or race as the subject to be diagnosed.

In some embodiments, subjects to be treated by the methods described herein can be a human patient suspected of having or at risk for asthma. In these embodiments, the methods described herein can provide prophylactic treatment. A subject suspected of having asthma or at risk for asthma may exhibit one or more common symptoms of asthma, such as those indicated above. Such a subject can also be identified by routine medical procedures. A subject at risk for asthma can be associated with one or more risk factors of asthma. Such risk factors include, but not limited to, family history of asthma (e.g., having a blood relative such as a parent or sibling, with asthma), other allergic conditions (e.g., such as atopic dermatitis or allergic rhinitis), overweight, smoking or exposure to secondhand smoke, exposure to exhaust fumes or other types of pollution, and exposure to occupational triggers, such as chemicals used in farming, hairdressing and manufacturing. Gender and age may also play roles in asthma development. For example, childhood asthma occurs more frequently in boys than in girls.

In some embodiments, a subject to be treated by the methods described herein can be a child who is 18 years old or younger, e.g., 6 months-18 years old, inclusive. In some embodiments, the subject may be a child at the age of 12 or over, e.g., 12-18 years old, inclusive. In some embodiments, the subject may be a child at the age of 5-11. In some embodiments, the subject may be a child under the age of 5, e.g., 6 months to 4 years old, inclusive.

In some embodiments, a subject to be treated by the methods described herein can be an adult who is over the age of 18, such as 19-80 years old, inclusive. In some embodiments, the subject is over the age of 21, e.g., 21-80 years old, inclusive. In some embodiments, an adult subject to be treated by the methods described herein may be an elderly who is over the age of 65, such as 66-80 years old.

A subject who needs the treatment as described herein can be identified via routine medical examination.

### III. Asthma Treatment

Any of the cysteamine and/or pharmaceutical compositions comprising the same, e.g., those described herein, can be administered to a subject in need thereof, e.g., those described herein, for treating asthma. For example, in some embodiments, the subject is a human patient at risk for asthma. In some embodiments, the subject is a human patient having asthma, for example, moderate to severe persistent asthma.

In some embodiments, a subject to be treated can be a child who is 18 years old or younger, e.g., 6 months-18 years old, inclusive. In some embodiments, the subject may be a child at the age of 12 or over, e.g., 12-18 years old, inclusive. In some embodiments, the subject may be a child at the age of 5-11. In some embodiments, the subject may be a child under the age of 5, e.g., 6 months to 4 years old, inclusive.

In some embodiments, a subject to be treated can be an adult who is over the age of 18, such as 19-80 years old, inclusive. In some embodiments, the subject is over the age of 21, e.g., 21-80 years old, inclusive. In some embodiments, an adult subject to be treated by the methods described herein may be an elderly who is over the age of 65, such as 66-80 years old.

The term “treating” or “treatment” as used herein refers to application or administration of cysteamine (e.g., in any suitable form as described herein) or a pharmaceutically acceptable salt thereof, a steroid treatment, or a combined treatment to a subject, who has asthma, a symptom of asthma, or a predisposition toward asthma, with the purpose to cure, heal, alleviate, relieve, alter, remedy, ameliorate, improve, or affect the disease, the symptoms of the disease, or the predisposition toward the disease.

In some embodiments, the treatment is prophylactic. The term “prophylactic” refers to application or administration of cysteamine (e.g., in any suitable form as described herein) or a pharmaceutically acceptable salt thereof, a steroid treatment, or a combined treatment to a subject who is at risk for asthma that prevents the occurrence, or delays the onset, of asthma. For example, a treatment is prophylactic when administration of cysteamine (e.g., in any suitable form as described herein) or a pharmaceutically acceptable salt thereof prevents development of asthma or delays the onset of asthma in a subject, e.g., a subject who is subsequently exposed to an allergen after the treatment.

In some embodiments, the treatment is therapeutic. The term “therapeutic” refers to application or administration of cysteamine (e.g., in any suitable form as described herein) or a pharmaceutically acceptable salt thereof, a steroid treatment, or a combined treatment to a subject, who has asthma or a symptom of asthma that improves at least one or more symptoms associated with asthma, e.g., reduced lung function, or reduced frequency of asthma attack. For example, a treatment is therapeutic when administration of cysteamine (e.g., in any suitable form as described herein) or a pharmaceutically acceptable salt thereof reduces the frequency of asthma attack or asthma exacerbation, e.g., induced by an allergen, in an asthmatic patient. Signs and symptoms of an asthma attack or asthma exacerbation may vary among individuals. Subjects having asthma exacerbation generally have their airways become swollen and inflamed, and thus they may cough, wheeze, and/or have trouble breathing. In some embodiments, subjects having asthma exacerbation may not respond to a quick-acting rescue medication or inhaler.

To perform the methods of treatment described herein, an effective amount of cysteamine or a pharmaceutical composition comprising the same can be administered to a subject in need of the treatment.

An “effective amount” refers to an amount of a cysteamine (e.g., in any suitable form as described herein) or a pharmaceutically acceptable salt thereof that alone, or together with further doses, produces the desired response, e.g., elimination or alleviation of symptoms, prevention or reduction the risk of asthma exacerbation, improvement of lung function, a reduction in frequency of night awakening, or usage of rescue medication, a reduction in frequency of asthma exacerbation (e.g., exercised-induced asthma exacerbation or allergen-induced asthma exacerbation) and/or restoration of quality of life. The desired response is to inhibit the progression or the symptoms of the disease. This may involve only slowing the progression of the disease temporarily, although it may involve halting the progression of the disease permanently. This can be monitored by routine methods. The desired response to treatment of the disease also can be delaying the onset or even preventing the onset of the disease.

Such amounts will depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

For example, an effective amount of cysteamine (e.g., in any suitable form as described herein) or a pharmaceutically acceptable salt thereof when administered to a subject in need thereof results in, e.g., by reducing the frequency of asthma exacerbation (e.g., induced by exercise or exposure to an allergen), by at least about 10% or more, including, e.g., at least about 20%, at least about 30%, at least about 40%, at least about 50%, at least about 60%, at least about 70%, at least about 80%, at least about 90% or more, as compared to the frequency of asthma exacerbation (e.g., induced by exercise or exposure to allergen) without administration of cysteamine or a pharmaceutically acceptable salt thereof.

In some embodiments, an effective amount of cysteamine (e.g., in any suitable form as described herein) or a pharmaceutically acceptable salt thereof when administered to a subject in need thereof results in, e.g., by reducing the frequency of night awakening, by at least about 10% or more, including, e.g., at least about 20%, at least about 30%, at least about 40%, at least about 50%, at least about 60%, at least about 70%, at least about 80%, at least about 90% or more, as compared to the frequency of night awakening without administration of cysteamine or a pharmaceutically acceptable salt thereof.

In some embodiments, an effective amount of cysteamine (e.g., in any suitable form as described herein) or a pharmaceutically acceptable salt thereof when administered to a subject in need thereof results in, e.g., by improving lung function (e.g., assessed using FEV₁ and/or FEV₁/FVC value determined by spirometry), by at least about 10% or more, including, e.g., at least about 20%, at least about 30%, at least about 40%, at least about 50%, at least about 60%, at least about 70%, at least about 80%, at least about 90% or more, as compared to the lung function without administration of cysteamine or a pharmaceutically acceptable salt thereof. In some embodiments, an effective amount of cysteamine or a pharmaceutically acceptable salt thereof when administered to a subject in need thereof results in a FEV₁ value and/or a FEV₁/FVC value (as determined by spirometry) that is within 20% (including, e.g., within 15%, within 10%, within 5%, or less) of a reference value. A reference value generally refers to a value obtained from a population of healthy normal subjects, e.g., at a similar age, height, weight, gender, and/or race as the subject to be diagnosed.

In some embodiments, an effective amount of cysteamine (e.g., in any suitable form as described herein) or a pharmaceutically acceptable salt thereof for use in the methods described herein can be equivalent to 2 mg/kg/day to 50 mg/kg/day of free base cysteamine, or 3 mg/kg/day to 40 mg/kg/day of free base cysteamine, or 4 mg/kg/day to 30 mg/kg/day of free base cysteamine, or 5 mg/kg/day to 25 mg/kg/day of free base cysteamine, or 6.25 mg/kg/day to 25 mg/kg/day of free base cysteamine, or 6.25 mg/kg/day to 12.5 mg/kg/day of free base cysteamine, or 5 mg/kg/day to 15 mg/kg/day of free base cysteamine, or 5 mg/kg/day to 12.5 mg/kg/day of free base cysteamine, or 0.5 mg/kg/day to 20 mg/kg/day of free base cysteamine, or 1 mg/kg/day to 15 mg/kg/day of free base cysteamine, or 1 mg/kg/day to 10 mg/kg/day of free base cysteamine, or 1 mg/kg/day to 5 mg/kg/day of free base cysteamine, or 0.5 mg/kg/day to 5 mg/kg/day of free base cysteamine.

In some embodiments where a subject in need of the treatment is over the age of 12 (e.g., 13-80 years old, inclusive), and/or over 50 kg (e.g., 51 kg-100 kg, inclusive) in body weight, an effective amount of cysteamine (e.g., in any suitable form as described herein) or a pharmaceutically acceptable salt thereof for administration to the subject can be equivalent to 200 mg/day to 500 mg/day of free base cysteamine (including, e.g., 200 mg/day to 450 mg/day of free base cysteamine, or 250 mg/day to 400 mg/day of free base cysteamine, or 300 mg/day to 450 mg/day of free base cysteamine).

In some embodiments where a subject in need of the treatment has a body weight in a range of 20 kg to 50 kg, an effective amount of cysteamine (e.g., in any suitable form as described herein) or a pharmaceutically acceptable salt thereof for administration to the subject can be equivalent to 120 mg/day to 450 mg/day of free base cysteamine (including, e.g., 120 mg/day to 400 mg/day of free base cysteamine, or 150 mg/day to 350 mg/day of free base cysteamine, or 200 mg/day to 400 mg/day of free base cysteamine).

In some embodiments where a subject in need of the treatment has a body weight under 20 kg, an effective amount of cysteamine (e.g., in any suitable form as described herein) or a pharmaceutically acceptable salt thereof for administration to the subject can be equivalent to 40 mg/day to 250 mg/day of free base cysteamine (including, e.g., 40 mg/day to 200 mg/day of free base cysteamine, or 60 mg/day to 250 mg/day of free base cysteamine, or 80 mg/day to 300 mg/day of free base cysteamine).

In some embodiments where a subject in need of the treatment has a body weight under 5 kg, an effective amount of cysteamine (e.g., in any suitable form as described herein) or a pharmaceutically acceptable salt thereof for administration to the subject can be equivalent to 40 mg/day to 100 mg/day of free base cysteamine (including, e.g., 40 mg/day to 80 mg/day of free base cysteamine, or 50 mg/day to 100 mg/day of free base cysteamine, or 60 mg/day to 100 mg/day of free base cysteamine).

In some embodiments where a subject in need of the treatment has a body weight in a range of 5 kg to 9 kg, an effective amount of cysteamine (e.g., in any suitable form as described herein) or a pharmaceutically acceptable salt thereof for administration to the subject can be equivalent to 60 mg/day to 150 mg/day of free base cysteamine (including, e.g., 60 mg/day to 120 mg/day of free base cysteamine, or 80 mg/day to 150 mg/day of free base cysteamine, or 100 mg/day to 150 mg/day of free base cysteamine).

In some embodiments where a subject in need of the treatment has a body weight in a range of 10 kg to 13 kg, an effective amount of cysteamine (e.g., in any suitable form as described herein) or a pharmaceutically acceptable salt thereof for administration to the subject can be equivalent to 80 mg/day to 200 mg/day of free base cysteamine (including, e.g., 80 mg/day to 150 mg/day of free base cysteamine, or 100 mg/day to 200 mg/day of free base cysteamine, or 120 mg/day to 200 mg/day of free base cysteamine).

In some embodiments where a subject in need of the treatment has a body weight in a range of 14 kg to 17 kg, an effective amount of cysteamine (e.g., in any suitable form as described herein) or a pharmaceutically acceptable salt thereof for administration to the subject can be equivalent to 100 mg/day to 250 mg/day of free base cysteamine (including, e.g., 100 mg/day to 200 mg/day of free base cysteamine, or 125 mg/day to 225 mg/day of free base cysteamine, or 150 mg/day to 250 mg/day of free base cysteamine).

In some embodiments where a subject in need of the treatment has a body weight in a range of 18 kg to 22 kg, an effective amount of cysteamine (e.g., in any suitable form as described herein) or a pharmaceutically acceptable salt thereof for administration to the subject can be equivalent to 120 mg/day to 300 mg/day of free base cysteamine (including, e.g., 120 mg/day to 250 mg/day of free base cysteamine, or 125 mg/day to 275 mg/day of free base cysteamine, or 150 mg/day to 300 mg/day of free base cysteamine).

In some embodiments where a subject in need of the treatment has a body weight in a range of 23 kg to 31 kg, an effective amount of cysteamine (e.g., in any suitable form as described herein) or a pharmaceutically acceptable salt thereof for administration to the subject can be equivalent to 140 mg/day to 350 mg/day of free base cysteamine (including, e.g., 140 mg/day to 300 mg/day of free base cysteamine, or 160 mg/day to 325 mg/day of free base cysteamine, or 180 mg/day to 350 mg/day of free base cysteamine).

In some embodiments where a subject in need of the treatment has a body weight in a range of 32 kg to 40 kg, an effective amount of cysteamine (e.g., in any suitable form as described herein) or a pharmaceutically acceptable salt thereof for administration to the subject can be equivalent to 160 mg/day to 400 mg/day of free base cysteamine (including, e.g., 160 mg/day to 350 mg/day of free base cysteamine, or 180 mg/day to 375 mg/day of free base cysteamine, or 200 mg/day to 400 mg/day of free base cysteamine).

In some embodiments where a subject in need of the treatment has a body weight in a range of 41 kg to 49 kg, an effective amount of cysteamine (e.g., in any suitable form as described herein) or a pharmaceutically acceptable salt thereof for administration to the subject can be equivalent to 180 mg/day to 450 mg/day of free base cysteamine (including, e.g., 180 mg/day to 400 mg/day of free base cysteamine, or 200 mg/day to 425 mg/day of free base cysteamine, or 250 mg/day to 450 mg/day of free base cysteamine).

In some embodiments, the daily effective amount of cysteamine (e.g., in any suitable form as described herein) or a pharmaceutically acceptable salt thereof, e.g., ones described herein, can be divided into multiple doses (e.g., 2-4 doses) for administration at given time intervals during the day. For example, in some embodiments, the daily effective amount of cysteamine (e.g., in any suitable form as described herein) or a pharmaceutically acceptable salt thereof, e.g., ones described herein, can be divided into 2 equal doses for a b.i.d. (twice a day) administration. In some embodiments, the daily effective amount of cysteamine (e.g., in any suitable form as described herein) or a pharmaceutically acceptable salt thereof, e.g., ones described herein, can be divided into 3 equal doses for a t.i.d. (three times a day) administration. In some embodiments, the daily effective amount of cysteamine (e.g., in any suitable form as described herein) or a pharmaceutically acceptable salt thereof, e.g., ones described herein, can be divided into 4 equal doses for a q.i.d (four times a day) administration.

In some embodiments, a subject in need thereof can be administered cysteamine (e.g., in any suitable form as described herein) or a pharmaceutically acceptable salt thereof four times a day, each administration in an amount of 10 mg or higher, including, e.g., 20 mg, 30 mg, 40 mg, 50 mg, 60 mg, 70 mg, 80 mg, 90 mg, 100 mg, 150 mg, 200 mg, or 250 mg.

In some embodiments, a subject in need thereof can be administered cysteamine (e.g., in any suitable form as described herein) or a pharmaceutically acceptable salt thereof two times a day, each administration in an amount of 20 mg or higher, including, e.g., 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, or 500 mg.

The cysteamine (e.g., in any suitable form as described herein) or pharmaceutically acceptable salt thereof can be administered to a subject in need thereof as a single anti-asthma agent or in combination with another anti-asthma agent. In some embodiments, the cysteamine (e.g., in any suitable form as described herein) or pharmaceutically acceptable salt thereof is administered to a subject in need thereof as a single anti-asthma agent, i.e., the subject is given cysteamine or a pharmaceutically acceptable salt thereof as the only anti-asthma agent, which is not co-used with other anti-asthma treatment.

In other embodiments, the cysteamine (e.g., in any suitable form as described herein) or pharmaceutically acceptable salt thereof is co-administered with a different anti-asthma agent. By “co-administered” or “in combination with” is meant that a subject is provided with cysteamine (e.g., in any suitable form as described herein) or a pharmaceutically acceptable salt thereof with a different anti-asthma agent during the course of treatment, such as concurrently, consecutively, intermittently, or in other regimens. Examples of additional anti-asthma agents include, but are not limited to bronchodilators, steroids, mast cell stabilizers, immunomodulators, long acting beta agonists, leukotrine modifiers or blockers, cromolyn, nedocromil, theophylline, and any combinations thereof.

In some embodiments, the cysteamine (e.g., in any suitable form as described herein) or pharmaceutically acceptable salt thereof is administered in combination with a steroid. Exemplary steroids include, but not limited to, prednisone, corticosteroid, methylprednisolone, dexamethasone, or a combination thereof. In some examples, a steroid is inhaled corticosteroid, including fluticasone (Flovent Diskus, Flovent HFA), budesonide (Pulmicort Flexhaler), mometasone (Asmanex), ciclesonide (Alvesco), flunisolide (Aerobid), and beclomethasone (Qvar). In some embodiments, the prednisone is applied by oral administration to the subject and/or the corticosteroid is applied by pulmonary administration.

When cysteamine or a pharmaceutically acceptable salt thereof is co-used with a second anti-asthma agent (e.g., those described herein), it may be formulated together with the second anti-asthma agent in a single pharmaceutical composition, which may be in any suitable form as described herein (e.g., enteric-coated or sustained release form for oral administration or in an injectable formulation). Alternatively, the cysteamine or a pharmaceutically acceptable salt thereof and the second anti-asthma agent may be formulated separately Administration of asthma treatment described herein may be accomplished by any method known in the art (see, e.g., Harrison's Principle of Internal Medicine, McGraw Hill Inc., 18^{th} ed., 2011). For combined treatment, each agent can be administered via the same route or different routes. Administration may be local or systemic. Administration may be, for example, parenteral (e.g., intravenous, intraperitoneal, subcutaneous, intra-arterial or intradermal), pulmonary (e.g., by inhalation through nose or mouth), or oral. Compositions for different routes of administration are well known in the art (see, e.g., Remington: The Science and Practice of Pharmacy, Pharmaceutical Press, 22^{nd} ed., 2012). The compositions may also be formulated as modified release dosage forms, including delayed-, extended-, prolonged-, sustained-, pulsed-, controlled-, accelerated- and fast-, targeted-, programmed-release, and gastric retention dosage forms. These dosage forms can be prepared according to conventional methods and techniques known to those skilled in the art. Dosage will depend the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Dosage can be determined by the skilled artisan.

In some embodiments, cysteamine (e.g., in any suitable form as described herein) or a pharmaceutically acceptable salt thereof and/or an anti-asthma agent can be administered orally. Oral administration also includes buccal, lingual, and sublingual administration. In some embodiments, pharmaceutical compositions comprising cysteamine (e.g., in any suitable form as described herein) or a pharmaceutically acceptable salt thereof may be provided in solid, semisolid, or liquid composition for oral administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

In some embodiments, cysteamine (e.g., in any suitable form as described herein) or a pharmaceutically acceptable salt thereof and/or an anti-asthma agent can be administered by injection (e.g., parenterally such as intravenously or intraperitoneally). Preparations for parenteral administration include sterile aqueous or non-aqueous solutions, suspensions, and emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain one or more of a preserving agent, a wetting agent, an emulsifying agent and a dispersing agent. The dosage forms may be sterilized by, for example, filtration of the composition, by irradiating the composition, or by heating the composition. They can also be manufactured using sterile water, or some other sterile injectable medium, prior to use.

In some embodiments, the route of administration of asthma treatment is pulmonary and can be delivered to the lungs by any number of means known in the art. In some embodiments, pulmonary formulations of the present invention are administered as aerosol compositions. Aerosol formulations are known to those skilled in the art and described, for example, in Remington: The Science and Practice of Pharmacy, supra. The aerosol formulation may be, for example, either a solution aerosol, in which the active agents are soluble in the carrier (e.g., propellant), or a dispersion aerosol, in which the active agents are suspended or dispersed throughout the carrier or carriers and optional solvent. In aerosol formulations, the carrier is typically a propellant, usually a liquefied gas or mixture of liquefied gases. For example, the carrier may be a fluorinated hydrocarbon. Exemplary fluorinated hydrocarbons include, but are not limited to, trichloromonofluoromethane, dichlorodifluoromethane, dichlorotetrafluoroethane, chloropentafluoroethane, 1-chloro-1,1-difluoroethane, 1,1-difluoroethane, octafluorocyclobutane, 1,1,1,2-tetrafluoroethane (HFA-134a), 1,1,1,2,3,3,3-heptafluoropropane (HFA-227) and combinations thereof. As is readily appreciated by one skilled in the art, the aerosol formulations of the invention may include one or more excipients. The aerosol formulations may, for example, contain: a solvent (e.g., water, ethanol and mixtures thereof) for increasing the solubility of the active agent; an antioxidant (e.g., ascorbic acid) for inhibiting oxidative degradation of the active agents; a dispersing agent (e.g., sorbitan trioleate, oleyl alcohol, oleic acid, lecithin, corn oil, and combinations thereof) for preventing agglomeration of particles; and/or a lubricant (e.g., isopropyl myristate) for providing slippage between particles and lubricating the components, e.g., the valve and spring, of an inhaler. Dry powder formulations for pulmonary delivery include the active agent and any carrier suitable for pulmonary drug administration. The carrier may be, for example, a pharmaceutical sugar such as fructose, galactose, glucose, lactitol, lactose, maltitol, maltose, mannitol, melezitose, myoinositol, palatinite, raffinose, stachyose, sucrose, trehalose, xylitol, hydrates thereof or combinations thereof. Selected components are initially combined and then blended to form a homogeneous, uniform powder mixture. Techniques for preparation of such powders are well known in the art. Regardless of technique employed the resulting powder is preferably both homogeneous and uniform. Typically, the active agents will make up from about 0.10% to about 99% (w/w) of the total formulation.

Pulmonary formulations of the may also be a liquid composition for inhalation, as well known in the art. See, e.g., Remington: The Science and Practice of Pharmacy, supra. Preferably, the liquid is an aqueous suspension, but aqueous solutions may also be used. The liquid formulations may include one or more carriers in addition to the active agents. Generally the carrier is a sodium chloride solution having a concentration making the formulation isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Combining the components followed by conventional mixing effects a liquid formulation suitable for inhalation. Typically, the active agents will make up from about 0.01% to about 40% of the total formulation.

Various known devices may be used to administer pulmonary formulations, whether dry powder, aerosol or liquid. Dry powder inhalers are well known to those skilled in the art and are used to administer the aforementioned dry powder formulations: Suitable dry powder inhalation devices for administering the present formulations include, for example, TURBUHALER® (Astra Pharmaceutical Products, Inc., Westborough, MA), ROTAHALER® (Allen & Hanburys, Ltd., London, England). Aerosol formulations may be administered via pressurized metered-dose inhalers. A metered-dose inhaler may automatically dispense, in a puff intended for inhalation in a single or multiple breaths, a set amount of a treatment described herein when activated by the subject in need of treatment. Liquid formulations of the invention may be administered via a pump spray bottle or nebulizer.

In some embodiments, the method further comprises taking actions other than or in addition to an asthma treatment described herein. In some embodiments, the method further comprises monitoring development of an asthma symptom of a subject who is at risk for asthma, or monitoring the effectiveness of the treatment. The monitoring may comprise a physical examination and/or spirometry. If the subject is not responsive to an administered dose of cysteamine or a pharmaceutically acceptable salt thereof, a physician can increase the dose of cysteamine or a pharmaceutically acceptable salt thereof, e.g., based on the medical and/or physical condition of the subject.

In some embodiments, the method further comprises performing a home intervention to reduce the risk for asthma development. Home intervention may involve reduce the level of exposure to certain matters that may induce asthma, e.g., mold, allergen, etc. In other embodiments, home intervention may involve dietary intervention, e.g., adding fatty acids such as linoleic acid to diet.

In some embodiments, the method further comprises reducing environmental risk factors for asthma development. Environmental risk factors refer to those that are likely to induce or enhance asthma. Examples include, but are not limited to, traffic pollution, allergens (e.g., pet allergens such as those from cat, dog, dust mite, pollen), smoke/tobacco exposure, mold exposure, ozone exposure, or NO₂ exposure.

### IV. Kits for Use in Asthma Treatment

Another aspect of the present disclosure relates to kits for use in asthma treatment described herein. Accordingly, in some embodiments, such a kit can comprise cysteamine (e.g., in any suitable form as described herein) or a pharmaceutically acceptable salt thereof, or a pharmaceutical composition comprising the same.

In some embodiments, the kit can comprise instructions for use in accordance with any of the methods described herein. The instructions can comprise a description of administration of cysteamine (e.g., in any suitable form as described herein) or a pharmaceutically acceptable salt thereof, or a pharmaceutical composition comprising the same, for asthma treatment. The instructions relating to cysteamine (e.g., in any suitable form as described herein) or a pharmaceutically acceptable salt thereof, or a pharmaceutical composition comprising the same, generally include information as to dosage, dosing schedule, and route of administration for the intended treatment. Such instructions may also include recommended weight-based dosages and/or age-based dosages.

Instructions supplied in the kits described herein are typically written instructions on a label or package insert (e.g., a paper sheet included in the kit), but machine-readable instructions (e.g., instructions carried on a magnetic or optical storage disk) are also acceptable. The label or package insert indicates that the composition is used for asthma treatment in subjects. In some embodiments, the label or package insert may indicate that the composition is suitable for use in specific groups of subjects, e.g., as described herein. Instructions may be provided for practicing any of the methods described herein.

Cysteamine (e.g., in any suitable form as described herein) or a pharmaceutically acceptable salt thereof, or a pharmaceutical composition comprising the same in the kit may be in suitable packaging. Suitable packaging includes, but is not limited to, vials, bottles, jars, flexible packaging (e.g., sealed Mylar or plastic bags), and the like. The packaging may be in unit doses, bulk packages (e.g., multi-dose packages) or sub-unit doses.

In some embodiments, the kit may further comprise an apparatus for delivering cysteamine (e.g., in any suitable form as described herein) or a pharmaceutically acceptable salt thereof, or a pharmaceutical composition comprising the same. Thus, also contemplated are packages for use in combination with a specific device, such as an inhaler, nasal administration device (e.g., an atomizer) or an infusion device such as a minipump. The container or packaging may have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Prophylactic Treatment With Cysteamine Significantly Prevents Asthma Development

Steroid non-responsive mice (e.g., vanin 1 (VNN1) knock-out (KO) mice) and normal mice (e.g., wild type (WT) BALB/c mice) were used in an experimental asthma model. Since VNN1 KO mice lack tissue cysteamine, these mice were used to determine whether replacement of cysteamine would be sufficient to restore responsiveness to a steroid treatment.

### Methods

### Treatment and Challenge Protocol

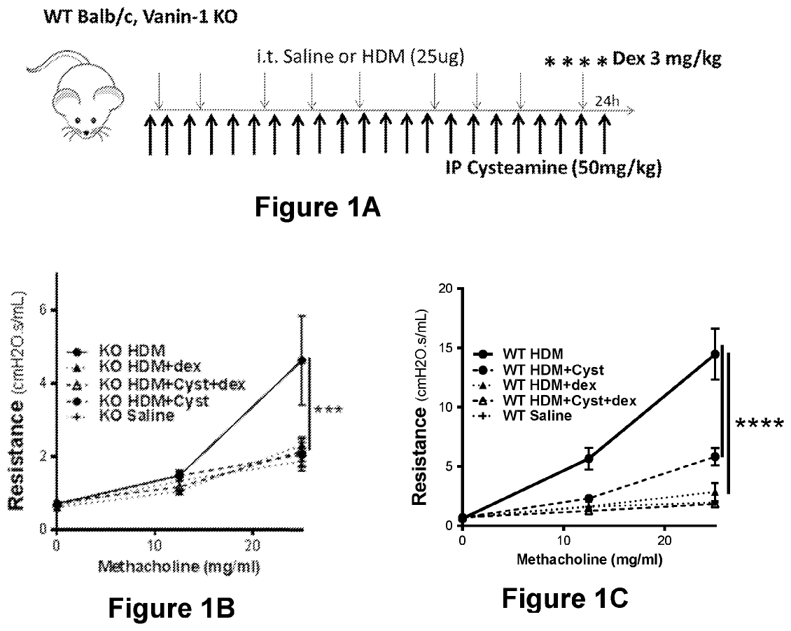

WT BALB/c and VNN1 KO mice were started on cysteamine replacement treatment (e.g., as illustrated in FIG. 1A) one day prior to the start of intratracheal (i.t.) challenges. Mice then received one intraperitoneal (i.p.) injection of cysteamine hydrochloride (50 mg/kg) or saline every day. Mice were challenged 3 times a week for 3 weeks to an allergen (e.g., house dust mite (HDM, 25 μg in 50 μl saline)) or saline. A subset of mice from each group received a corticosteroid (e.g., dexamethasone (3 mg/kg)) treatment during the last 4 days of the experiment.

### Airway Responsiveness Measurement

To determine the effectiveness of the treatment, airway responsiveness was measured as follows. 24 hours after the last challenge, invasive measurements were made with the FlexiVent apparatus (SCIREQ). Mice were anesthetized with ketamine, xylazine, and acepromazine (100, 20, and 10 mg/mL, respectively, mixed at a ratio of 4:1:1). Mouse tracheas were cannulated with a 19-gauge blunt needle, and the mice were ventilated at 150 breaths/min and 3.0 cm H₂O positive end-expiratory pressure. Two total lung capacity perturbations were then performed for airway recruitment before baseline measurement, and subsequent methacholine challenges were performed. Dynamic resistance was assessed after exposure to increasing concentrations of aerosolized methacholine (0, 12.5 and 25 mg/mL). The average of the 3 highest dynamic resistance values with a coefficient of determination of 0.9 or greater (as determined by using FlexiVent software) was used to determine the dose-response curve.

### Results

Cysteamine prophylactic treatment significantly prevented asthma in VNN1 KO and normal mice (FIG. 1B-1C). Treatment with cysteamine or cystamine blocked airway hyper-responsiveness (AHR) development in mice.

### Example 2: Efficacy of Cysteamine Therapeutic Treatment for Asthma

As shown in Example 1, cysteamine replacement decreased asthma in both WT and VNN1 KO mice. It was next sought to evaluate the effectiveness of cysteamine treatment after asthma is already established in normal mice (e.g., WT BALB/c mice).

### Methods

### Treatment and Challenge Protocol

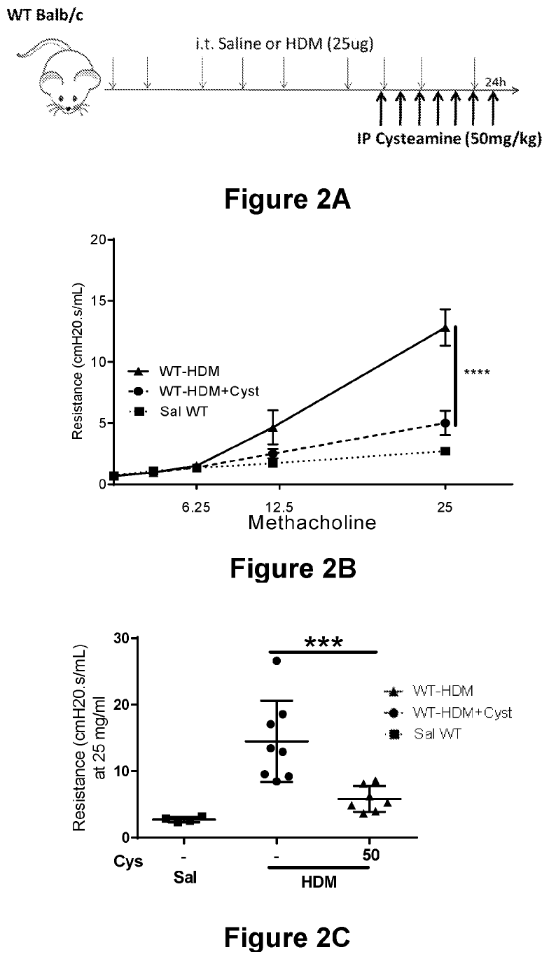

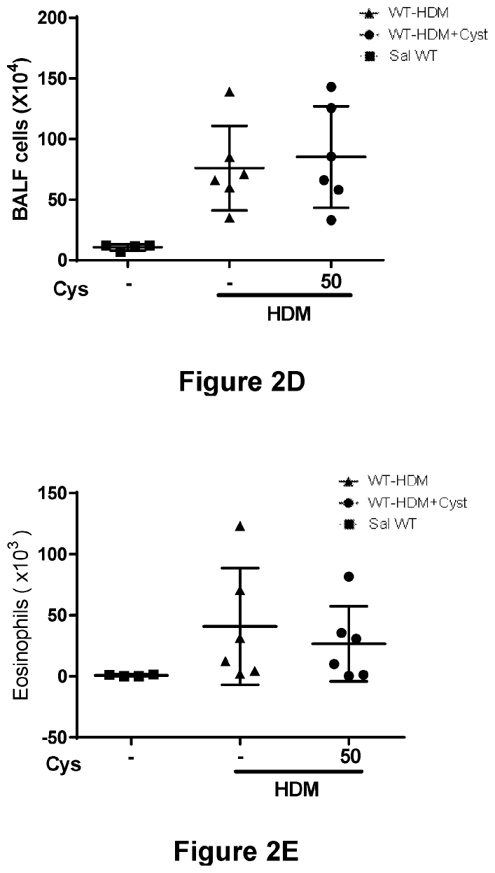

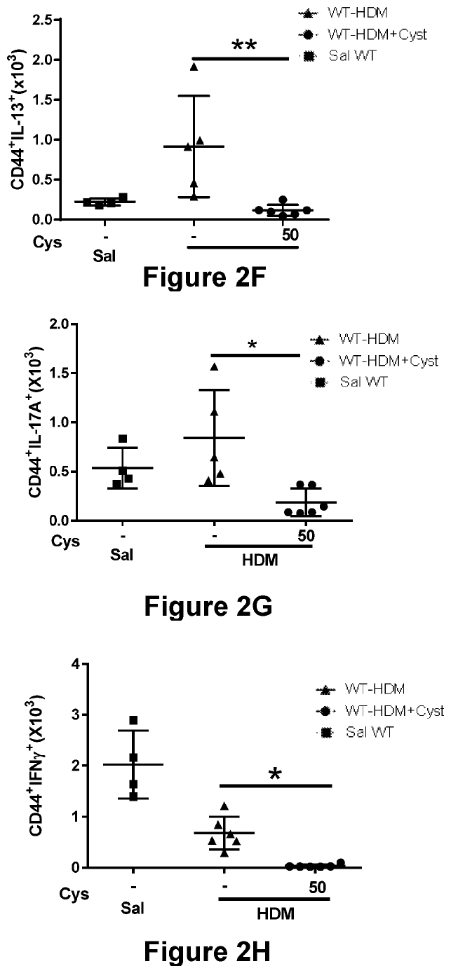

WT BALB/c mice underwent 6 intratracheal challenges to an allergen (e.g., HDM (25 μg)) to induce asthma development. Control mice underwent 6 intratracheal challenges to saline instead. The next day a subset of mice received cysteamine (50 mg/kg) treatment for seven days (on challenge days, mice received treatment 30 minutes prior to challenge). See FIG. 2A for the treatment and challenge protocol. The mice were sacrificed 24 hours after the last challenge.

### Airway Responsiveness Measurement

To determine the effectiveness of the treatment, airway responsiveness was measured as follows. 24 hours after the last challenge, invasive measurements were made with the FlexiVent apparatus (SCIREQ). Dynamic resistance was assessed after exposure to increasing concentrations of aerosolized methacholine (0, 3.125, 6.25, 12.5, and 25 mg/kg).

### Bronchoalveolar Lavage Fluid (BALF) Collection and Analysis

Bronchoalveolar lavage was performed by means of tracheal cannulation. Lungs were lavaged with 1 mL of 1×HBSS. The collected BALF was centrifuged, and total cell numbers were counted with a hemocytometer. Cells were spun onto slides and stained with the HEMA3 stain set. After the slides were cover slipped and de-identified, 200 cells were counted, and the total number of each cell type was calculated.

### Effector T-cell Analysis

To observe Th2, Th17, and Th1 cytokine production, lung cells were ex vivo stimulated with phorbol 12-myristate 13-acetate (PMA)+ionomycin (0.05 μg/mL and 0.50 m/mL, respectively) for an hour prior to the addition of Brefeldin-A for an additional 2.5 hours. Cells were stained with combinations of CD4-FITC, γδT-cell receptor-PE/Cy7, CD3-AF700, CD44-Pacific Blue, and Live/dead-BV510. After fixation and permeabilization cells were intracellularly stained with IL-13-PE, IL-17A-AF647, IFN-γ-PerCP5.5. Image an data acquisition was done on a FACSCanto III and analyzed with FlowJo software.

### Results

Cysteamine treatment was shown to significantly decrease asthma occurrence in mice with asthma. Treatment with cysteamine blocked airway hyper-responsiveness (AHR) development (FIG. 2B-2C) and reduced airway inflammation as demonstrated by a reduction in the presence of pathogenic T effector populations (Th2/Th17) in the lungs of mice with asthma (FIGS. 2F-2H). Cysteamine treatment also decreased the presence of eosinophils and neutrophils in the bronchoalveolar lavage fluid. (FIGS. 2D-2E)

### Example 3: Evaluation of Effectiveness of Cysteamine at Lower Doses to block Allergen-Induced Asthma Exacerbation

Since 50 mg/kg cysteamine treatment in Example 2 proved effective after 6 i.t. challenges, it was next sought to identify a minimal dose required to achieve a desired clinical effect such that dose-related side-effects of cysteamine can be minimized.

### Methods

### Treatment and Challenge Protocol

As shown in FIG. 3A, WT BALB/c mice underwent 9 intratracheal challenges to an allergen (e.g., HDM (25 μg)) over 3 weeks to induce asthma development. Control mice underwent 9 intratracheal challenges to saline instead. Once asthma was established, a subset of mice received cysteamine (e.g., Cystagon®, which corresponds to cysteamine bitartrate) at various doses (6.25, 12.5, 25, or 50 mg/kg) or saline vehicle intraperitoneal (i.p.) treatment 3 times every day for 2 weeks. On the 14th day, all mice received a single HDM recall challenge. Mice received scheduled i.p. treatment prior to airway responsiveness measurement.

### Airway Responsiveness Measurement

To determine the effectiveness of the treatment, airway responsiveness was measured as follows. 24 hours after the last challenge invasive measurements were made with the FlexiVent apparatus (SCIREQ). Dynamic resistance was assessed after exposure to increasing concentrations of aerosolized methacholine (0, 6.25, 12.5, 25, and 50 mg/kg).

### Effector T-cell Analysis

To observe Th2/Th17 cytokine production, lung cells were ex vivo stimulated with phorbol 12-myristate 13-acetate (PMA)+ionomycin (0.05 μg/mL and 0.50 μg/mL, respectively) for an hour prior to the addition of Brefeldin-A for an additional 2.5 hours. Cells were stained with combinations of CD4-FITC, γδT-cell receptor-PE/Cy7, CD3-AF700, CD44-Pacific Blue, and Live/dead-BV510. After fixation and premeabilization cells were intracellularly stained with IL-13-PE, IL-17A-AF647, IFN-γ-PerCP5.5. Image and data acquisition was done on a FACSCanto III and analyzed with FlowJo software.

### Results

Cysteamine (e.g., Cystagon®) was effective at blocking AHR in mice with asthma (FIGS. 3B-3C). The 6.25 mg/kg dose was effective at reducing AHR, but maximal effectiveness was achieved at 12.5 mg/kg/day dose—one fourth of the dose generally required for treatment of cystinosis. However, given the short half-life of cysteamine, a q.i.d (four times a time) dosing schedule with 6.25 mg/kg in each dose may be more effective. Cysteamine treatment (e.g., by administration of Cystagon®) also reduced airway inflammation (FIG. 3D).

In summary, two weeks of cysteamine (e.g., Cystagon®) treatment significantly decreased AHR and airway inflammation in asthmatic mice. Cysteamine treatment at 6.25, 12.5, and 50 mg/kg significantly decreased the presence of eosinophils and neutrophils in the bronchoalveolar lavage fluid. Cysteamine treatment significantly decreased the presence of pathogenic T effector populations (Th2/Th17) in the lungs of mice with asthma.

### Example 4: Drug Kinetics of Cysteamine

As shown in FIGS. 4A-4B, the cysteamine concentration rapidly degraded in mouse serum in the time after infusion ended (FIGS. 4A-4B).

### Example 5: Comparison of the Efficacy of Cysteamine vs. Steroid Such as Corticosteroids in Treatment of Asthma

Corticosteroids are currently the most prescribed treatment for asthma. This study compares the efficacy of cysteamine and steroid (e.g., corticosteroids) in treatment of asthma.

### Methods

### Treatment and Challenge Protocol

WT BALB/c mice underwent 6 intratracheal challenges to an allergen (e.g., HDM (25 μg)) to induce asthma development. Control mice underwent 6 intratracheal challenges to saline instead. When asthma was established, a subset of mice received cysteamine (50 mg/kg) treatment for seven days (on challenge days, mice received treatment 30 minutes prior to challenge). A subset of mice received corticosteroid (e.g., dexamethasone) treatment (3 mg/kg) during the last four days.

### Airway Responsiveness Measurement

To determine the effectiveness of the treatment, airway responsiveness was measured as follows. 24 hours after the last challenge, invasive measurements were made with the FlexiVent apparatus (SCIREQ). Dynamic resistance was assessed after exposure to increasing concentrations of aerosolized methacholine (0, 12.5, and 50 mg/kg).

### Effector T-cell Analysis

To observe Th2/Th17 cytokine production, lung cells were ex vivo stimulated with phorbol 12-myristate 13-acetate (PMA)+ionomycin (0.05 μg/mL and 0.50 m/mL, respectively) for an hour prior to the addition of Brefeldin-A for an additional two and a half hours. Cells were stained with combinations of CD4-FITC, γδT-cell receptor-PE/Cy7, CD3-AF700, CD44-Pacific Blue, and Live/dead-BV510. After fixation and premeabilization cells were intracellularly stained with IL-13-PE, IL-17A-AF647, IFN-γ-PerCP5.5. Image and data acquisition was done on a FACSCanto III and analyzed with FlowJo software.

### Results

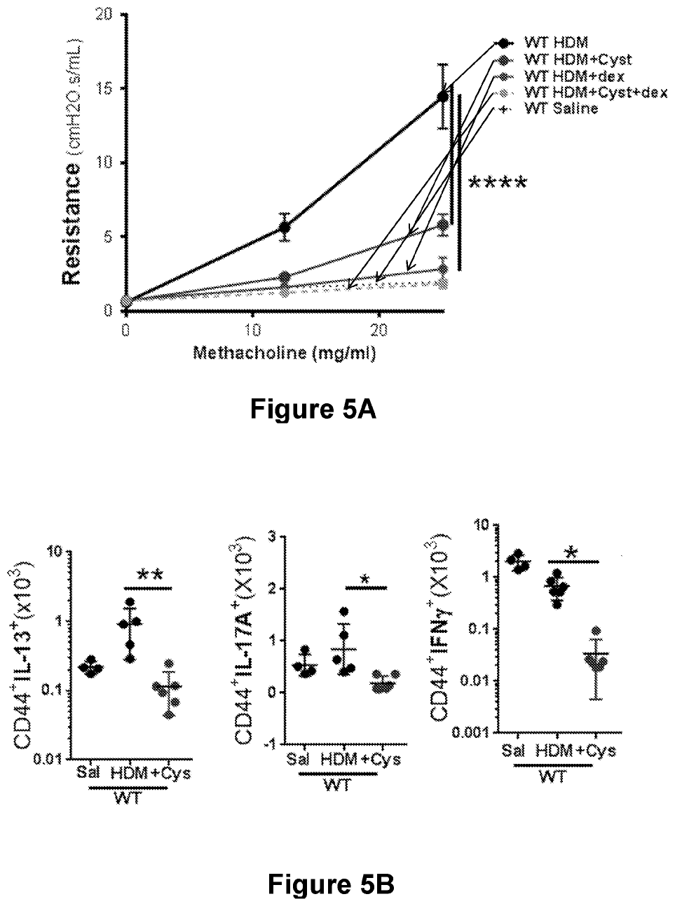

There were no significant differences between the cysteamine and dexamethasone treatment groups. Cysteamine and dexamethasone treatment decreased AHR in WT mice with asthma (FIG. 5A). Cysteamine treatment significantly decreased the presence of Th1, Th2, and Th17 cells present in the lungs of WT mice (FIG. 5B).

### Example 6: Efficacy of Oral Cysteamine Treatment in Asthma Management

Examples 3-4 show that i.p. administration of cysteamine can effectively block allergen-induced asthma exacerbations. It was next sought to determine if oral administration of cysteamine would provide the same efficacy in an asthma model as when cysteamine was administered i.p.

### Methods

### Treatment and Challenge Protocol

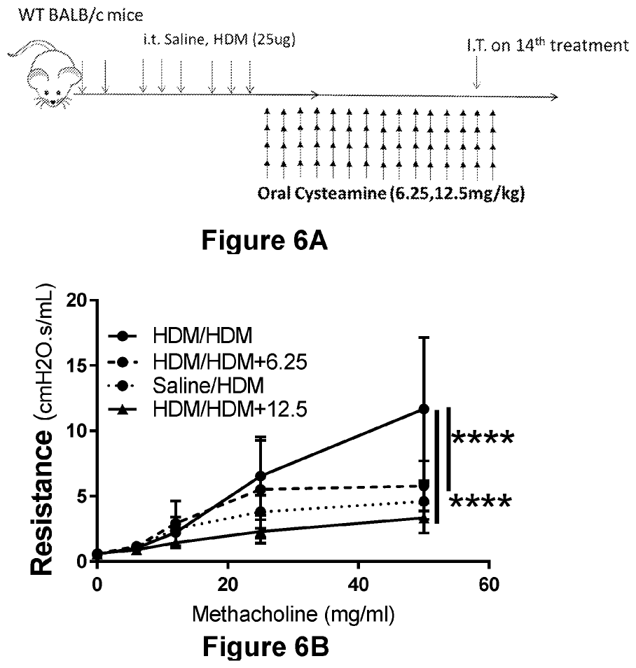

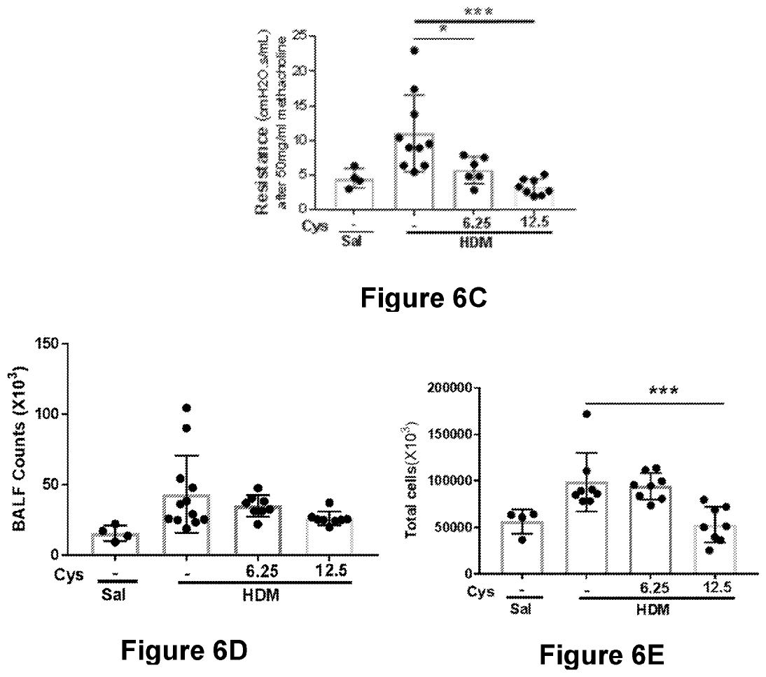

As illustrated in FIG. 6A, WT BALB/c mice underwent 9 intratracheal challenges to an allergen (e.g., HDM (25 μg)) to induce asthma development. Control mice underwent 9 intratracheal challenges to saline instead. Once asthma was established, a subset of mice received cysteamine (6.25 or 12.5 mg/kg) or saline vehicle oral treatment 4 times a day for 2 weeks. On the 14th day, all mice received a single HDM recall challenge. Mice received scheduled oral treatment prior to airway responsiveness measurement.

### Airway Responsiveness Measurement

To determine the effectiveness of the treatment, airway responsiveness was measured as follows. 24 hours after the last challenge invasive measurements were made with the FlexiVent apparatus (SCIREQ). Dynamic resistance was assessed after exposure to increasing concentrations of aerosolized methacholine (0, 6.25, 12.5, 25, and 50 mg/kg).

### Bronchoalveolar Lavage Fluid (BALF) Collection

Bronchoalveolar lavage was performed by means of tracheal cannulation. Lungs were lavaged with 1 mL of 1×HBSS. The collected BALF was centrifuged, and total cell numbers counted with a hemocytometer.

### Lung Cell Collection

Lungs were removed, and the upper right lobe was minced and incubated at 37° C. for 30 minutes in 1 mL of RPMI 1640 containing Liberase TL (0.5 mg/mL) and DNAse I (0.5 mg/mL). Lung cells were passed through a 70-μm cell strainer with a syringe rubber, and the strainer was washed with 5 mL of RPMI plus DNAse I media. Cells were centrifuged and re-suspended in 1 mL of PBS plus 0.5% BSA plus 2 mmol/L EDTA before being counted on a hemocytometer.

### Results

Cysteamine treatment decreased AHR (FIGS. 6B-6C) and airway inflammation (FIGS. 6D-6E) in WT mice with asthma.

### Example 7: Comparison of Cysteamine Dose in Asthma Treatment vs. Cystinosis Treatment

Treatment of cystinosis with cysteamine bitartrate (e.g., Cystagon®) is generally started at one fourth of a maintenance dose and then titrated up to therapeutic level of treatment. For example, children may receive 1.3 g/m² of cysteamine free base per day, divided into 4 doses. Adults or children greater than twelve years old and weighing more than one hundred and ten pounds may receive 2.0 g of cysteamine free base per day, divided into 4 doses. Up to 1.95 g/m² of cysteamine free base per day can be administered for treatment of cystinosis if necessary. For treatment of cystinosis with enteric-coated cysteamine bitartrate, the therapeutic dose is based on an equivalent amount of free base cysteamine as provided in cysteamine bitartrate but is administered in two 12-hour doses.

For treatment of cystinosis, an average six-year-old child (e.g., a body weight of 50 pounds or 22.7 kg with a body surface area of 0.8 M²) is given 1.6 g cysteamine per day.

For treatment of asthma, a dose of 50 mg/kg/day cysteamine (a dose used in the studies described herein) in an average six-year-old child (e.g., with a body weight of 22.7 kg) is equivalent to 1.135 g cysteamine per day, which is similar to the 1.6 g dose for treatment of cystinosis. However, as shown in the Examples 3 and 6, cysteamine at a dose of 12.5 mg/kg/day is effective to reduce asthma symptoms or occurrence.

### Example 8: Determining the Dose for Randomized Controlled Trials

### Methods

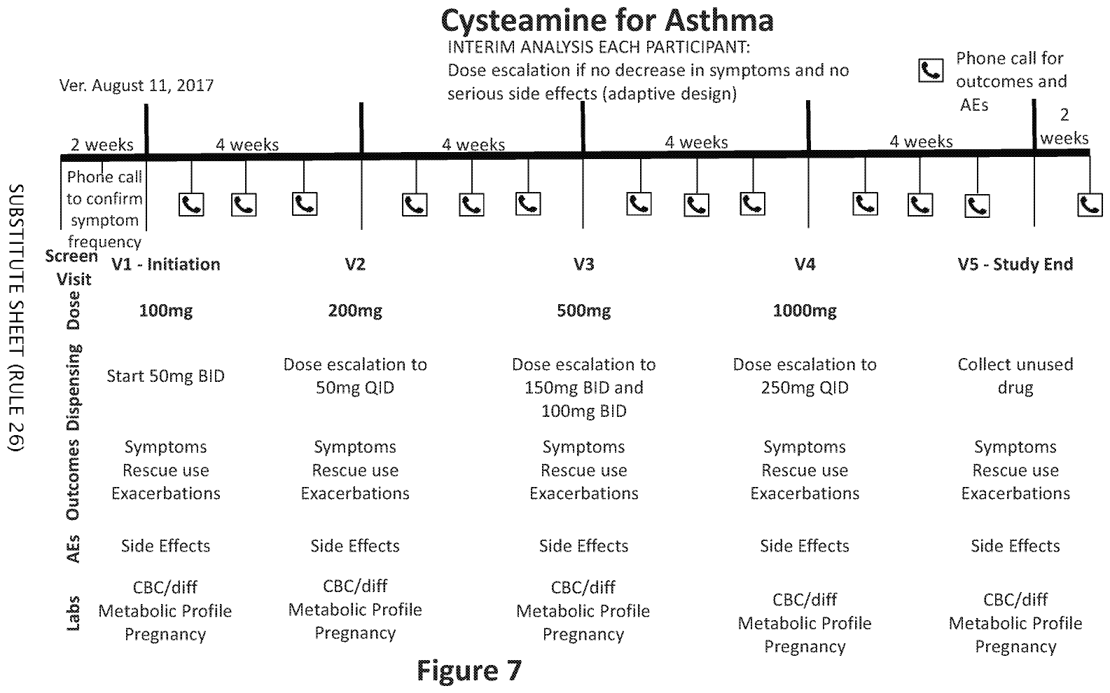

A sample size of 10 adults with persistent uncontrolled asthma are enrolled in this study. They are administered cysteamine (e.g., in any suitable form as described herein) according to an adaptive study design with dose escalation schedule as shown in FIG. 7. Side effects (e.g., nausea, dyspepsia, rash, bruising or streaks on the skin, and/or central nervous system side-effects) and efficacy (e.g., symptom frequency, exacerbation frequency, and/or frequency of rescue medication usage) of cysteamine treatment in asthmatic patients are determined and analyzed.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating asthma or reducing the risk of asthma occurrence, the method comprising: administering to a subject in need thereof an effective amount of cysteamine or a pharmaceutically acceptable salt thereof, wherein the subject is a human patient at risk for asthma or a human patient having moderate to severe persistent asthma.

2. The method of claim 1, wherein the subject is a human patient at risk for asthma and the method is for prophylactic treatment.

3. The method of claim 1, wherein the subject is a human patient having moderate to severe persistent asthma.

4. The method of claim 3, wherein the cysteamine or the pharmaceutically acceptable salt thereof is administered to the human patient in an amount sufficient to reduce allergen-induced asthma exacerbation.

5. The method of claim 1, wherein the cysteamine or the pharmaceutically acceptable salt thereof is administered to the subject as a single anti-asthma agent.

6. The method of claim 1, wherein the cysteamine or the pharmaceutically acceptable salt thereof is administered in combination with a steroid.

7. The method of claim 1, wherein the cysteamine or the pharmaceutically acceptable salt thereof is administered to the subject orally or by injection.

8. The method of claim 7, wherein the cysteamine or the pharmaceutically acceptable salt thereof is formulated in a pharmaceutical formulation, which is in an enteric-coated solid form or in a sustained-release form.

9. The method of claim 1, wherein the subject is an adult.

10. The method of claim 1, wherein the subject is a child at the age of 12 or over, a child at the age of 5-11, or a child under the age of 5.

11. The method of claim 1, wherein the effective amount of the cysteamine or the pharmaceutically acceptable salt thereof is equivalent to 5 mg/kg/day to 25 mg/kg/day of free base cysteamine.

12. The method of claim 1, wherein the effective amount of the cysteamine or the pharmaceutically acceptable salt thereof is equivalent to 6.25 mg/kg/day to 25 mg/kg/day of free base cysteamine.

13. The method of claim 1, wherein the effective amount of the cysteamine or the pharmaceutically acceptable salt thereof is equivalent to 5 mg/kg/day to 15 mg/kg/day of free base cysteamine.

14. The method of claim 1, wherein the effective amount of the cysteamine or the pharmaceutically acceptable salt thereof is equivalent to:
(i) 200 mg/day to 500 mg/day of free base cysteamine for a subject over the age of 12 and/or over 50 kg in body weight; (ii) 120 mg/day to 450 mg/day of free base cysteamine for a subject having a body weight in a range of 20 kg to 50 kg; and (iii) 40 mg/day to 250 mg/day of free base cysteamine for a subject having a body weight under 20 kg.

15. The method of claim 1, wherein the cysteamine or the pharmaceutically acceptable salt thereof is administered to the subject 2-4 times per day.

16. The method of claim 1, wherein the pharmaceutically acceptable salt of cysteamine is cysteamine bitartrate or cysteamine hydrochloride.

17. The method of claim 1, wherein the cysteamine is in a disulfide form or in a free base form.

18. A method for treating asthma, the method comprising: administering to a subject in need thereof cysteamine or a pharmaceutically acceptable salt thereof in an amount equivalent to 5 mg/kg/day to 25 mg/kg/day of free base cysteamine.

19. The method of claim 18, wherein the amount of the cysteamine or the pharmaceutically acceptable salt is equivalent to 6.25 mg/kg/day to 25 mg/kg/day of free base cysteamine.

20. The method of claim 18, wherein the amount of the cysteamine or the pharmaceutically acceptable salt thereof is equivalent to: (i) 200 mg/day to 500 mg/day of free base cysteamine for a subject over the age of 12 and/or over 50 kg in weight; (ii) 120 mg/day to 450 mg/day of free base cysteamine for a subject having a weight in a range of 20 kg to 50 kg; and (iii) 40 mg/day to 250 mg/day of free base cysteamine for a subject having a body weight under 20 kg.

21. The method of claim 18, wherein the cysteamine or the pharmaceutically acceptable salt thereof is administered to the subject 2-4 times per day.

22. The method of claim 18, wherein the subject is a human patient at risk of developing asthma, or suspected of having or having asthma.

23. The method of claim 22, wherein the subject is a human patient having moderate to severe persistent asthma.

24. The method of claim 18, wherein the cysteamine or the pharmaceutical acceptable salt thereof is administered to the subject as a single anti-asthma agent.

25. The method of claim 18, wherein the cysteamine or the pharmaceutically acceptable salt thereof is administered to the subject in combination with a steroid.

26. The method of claim 18, wherein the cysteamine or the pharmaceutically acceptable salt thereof is administered to the subject orally or by injection.

27. The method of claim 26, wherein the cysteamine or the pharmaceutically acceptable salt thereof is formulated in a pharmaceutical formulation, which is in an enteric-coated solid form or in a sustained-release form.

28. The method of claim 18, wherein the pharmaceutically acceptable salt of cysteamine is cysteamine bitartrate or cysteamine hydrochloride.

29. The method of claim 18, wherein the cysteamine is in a disulfide form or in a free base form.
